(19) United States
(12) Patent Application Publication
(10) Pub. No.: US 2024/0407705 A1
(43) Pub. Date: Dec. 12, 2024

(54) SHAPED DILATOR FOR TRANSSEPTAL PUNCTURE AND ELECTROANATOMICAL MAPPING

(71) Applicant: Boston Scientific Scimed, Inc., Maple Grove, MN (US)

(72) Inventors: Christian Balkovec, Cambridge (CA); Charlene Leung, Scarborough (CA); Zion Maynard, Maple Grove, MN (US)

(21) Appl. No.: 18/680,244

(22) Filed: May 31, 2024

Related U.S. Application Data
(60) Provisional application No. 63/507,173, filed on Jun. 9, 2023.

(51) Int. Cl. A61B 5/367; A61B 5/00; A61B 5/287; A61B 17/34

(52) U.S. Cl. CPC A61B 5/367; A61B 5/287; A61B 5/6855; A61B 17/3417

(57) ABSTRACT

A medical system includes a sheath and a dilator. The sheath includes an elongate body having a proximal portion and a distal portion, and a lumen extending from the proximal portion to the distal portion. The dilator is movable within the lumen. The dilator includes an elongate body having a proximal portion, a pre-formed distal portion, and a tapered distal tip. One or more electroanatomical mapping electrodes are located on the pre-formed distal portion. The one or more electroanatomical mapping electrodes is configured to electrically couple with an electroanatomical mapping system. The pre-formed distal portion has a substantially linear configuration when confined in the sheath, and an arcuate configuration when unconfined.

### CROSS REFERENCE TO RELATED APPLICATIONS

The present application claims priority to U.S. Provisional Patent Application No. 63/507,173 entitled “SHAPED DILATOR FOR TRANSSEPTAL PUNCTURE AND ELECTROANATOMICAL MAPPING,” filed Jun. 9, 2023, the entirety of which is hereby incorporated in its entirety.

### TECHNICAL FIELD

The present invention relates generally to methods and devices usable within the body of a patient. More specifically, the present invention is concerned with an apparatus and method for creating electroanatomical maps of heart structures using an electroanatomical mapping enabled dilator that can also be used to perform a transseptal puncture.

### BACKGROUND

Electroanatomical mapping (EAM) is an increasingly prevalent technology useful during in vivo procedures. It enables physicians to identify anatomical regions of the heart and patterns of electrical activation. This is especially useful when treating arrhythmias. Devices that are compatible with EAM systems allow operators to localize them and more easily target specific regions for treatment, allowing for better workflow, better treatment efficacy, and shorter procedure time.

With an increased effort within cardiac catheterization labs to reduce fluoroscopy, EAM has played an increasingly prominent role. To perform a transseptal puncture without fluoroscopy, physicians may create a quick map of the right atrium in conjunction with using ultrasound-based (such imaging as intracardiac echocardiography). To create a quick map of the right atrium, a user currently inserts a device into the patient's body that is capable of integrating with an EAM system to create a map representative of the patient's right atrial anatomy. Once the map of the right atrium is created, a user needs to remove the mapping device and insert a device or series of devices capable of performing a transseptal puncture. Repeated device insertions and removals is inefficient from both a workflow efficiency and safety standpoint, as each insertion/removal adds time to the procedure and creates the potential to introduce air and/or particulate into the patient's bloodstream.

### SUMMARY

Example 1 is a medical system including a sheath and a dilator. The sheath includes an elongate body having a proximal portion and a distal portion, and a lumen extending from the proximal portion to the distal portion. The dilator is movable within the lumen. The dilator includes an elongate body having a proximal portion, a pre-formed distal portion, and a tapered distal tip. One or more electroanatomical mapping electrodes are located on the pre-formed distal portion. The one or more electroanatomical mapping electrodes is configured to electrically couple with an electroanatomical mapping system. The pre-formed distal portion has a substantially linear configuration when confined in the sheath, and an arcuate configuration when unconfined.

Example 2 is the system of Example 1 wherein the sheath or the dilator includes a steering mechanism.

Example 3 is the system of any of Examples 1 or 2 wherein the pre-formed distal portion includes a first curve, a second curve, and a medial segment between the first curve and the second curve, and wherein an axis of the tapered distal tip is offset from an axis of the medial segment when the pre-formed distal portion is in the arcuate configuration.

Example 4 is the system of any of Examples 1-3 wherein the pre-formed distal portion and the tapered distal tip are located in the same plane when the pre-formed distal portion is in the arcuate configuration.

Example 5 is the system of any of Examples 1-3 wherein the pre-formed distal portion and the tapered distal tip are located in different planes when the pre-formed distal portion is in the arcuate configuration.

Example 6 is the system of any of Examples 1-5 wherein the sheath has a rigidity greater than a rigidity of the pre-formed distal portion.

Example 7 is the system of any of Examples 1-6 further comprising a guiding member configured to be inserted into a lumen of the dilator.

Example 8 is the system of any of Examples 1-7 further comprising a piercing member configured to be inserted into a lumen of the dilator.

Example 9 is the system of Example 8 wherein the piercing member is RF perforation device or a needle.

Example 10 is the system of any of Examples 1-9 wherein one or more conductor extends from the one or more electroanatomical mapping electrodes to the proximal portion of the dilator elongate body.

Example 11 is the system of any of Examples 1-10 wherein the tapered distal tip tapers from an outer diameter of approximately 0.111″ to approximately 0.060″ over a length of approximately 10 mm.

Example 12 is the system of any of Examples 1-11 wherein the dilator is formed of one or more low density polyethylene, high density polyethylene, shape memory polymer, or shape memory metal.

Example 13 is the system of any of Examples 1-12 further comprising a proximal electrode located proximal of the pre-formed distal portion that is configured to identify a stem of the dilator on the electroanatomical mapping system.

Example 14 is the system of any of Examples 1-13 wherein the electroanatomical mapping system comprises a display for displaying one or more anatomical images, parameters, and positioning information.

Example 15 is the system of any of Examples 1-14 wherein the one or more electroanatomical mapping electrodes includes three electrodes.

Example 16 is a medical system including a sheath and a dilator. The sheath includes an elongate body having a proximal portion and a distal portion, and a lumen extending from the proximal portion to the distal portion. The dilator is movable within the lumen. The dilator includes an elongate body having a proximal portion, a pre-formed distal portion, a tapered distal tip, and a lumen extending from the proximal portion to the tapered distal tip. One or more electroanatomical mapping electrodes is located on the pre-formed distal portion. The one or more electroanatomical mapping electrodes is configured to electrically couple with an electroanatomical mapping system. The pre-formed distal portion has a substantially linear configuration when confined in the sheath, and an arcuate configuration when unconfined.

Example 17 is the system of Example 16 wherein the sheath or the dilator includes a steering mechanism.

Example 18 is the system of Example 16 wherein the pre-formed distal portion includes a first curve, a second curve, and a medial segment between the first curve and the second curve, and wherein an axis of the tapered distal tip is offset from an axis of the medial segment when the pre-formed distal portion is in the arcuate configuration.

Example 19 is the system of Example 16 wherein the pre-formed distal portion and the tapered distal tip are located in the same plane when the pre-formed distal portion is in the arcuate configuration.

Example 20 is the system of Example 16 wherein the pre-formed distal portion and the tapered distal tip are located in different planes when the pre-formed distal portion is in the arcuate configuration.

Example 21 is the system of Example 16 wherein the sheath has a rigidity greater than a rigidity of the pre-formed distal portion.

Example 22 is the system of Example 16 further comprising a guiding member configured to be inserted into the lumen of the dilator.

Example 23 is the system of Example 16 further comprising a piercing member configured to be inserted into the lumen of the dilator.

Example 24 is the system of Example 23 wherein the piercing member is RF perforation device or a needle.

Example 25 is the system of Example 16 wherein one or more conductor extends from the one or more electroanatomical mapping electrodes to the proximal portion of the dilator elongate body.

Example 26 is the system of Example 16 wherein the tapered distal tip tapers from an outer diameter of approximately 0.111″ to approximately 0.060″ over a length of approximately 10 mm.

Example 27 is the system of Example 16 wherein the dilator is formed of one or more low density polyethylene, high density polyethylene, shape memory polymer, or shape memory metal.

Example 28 is the system of Example 16 further comprising a proximal electrode located proximal of the pre-formed distal portion that is configured to identify a stem of the dilator on the electroanatomical mapping system.

Example 29 is the system of Example 16 wherein the electroanatomical mapping system comprises a display for displaying one or more anatomical images, parameters, and positioning information.

Example 30 is the system of Example 16 wherein the one or more electroanatomical mapping electrodes includes three electrodes.

Example 31 is a medical system including a sheath and a dilator. The sheath includes an elongate body having a proximal portion and a distal portion, and a lumen extending from the proximal portion to the distal portion. The dilator is movable within the lumen. The dilator includes an elongate body having a proximal portion, a pre-formed distal portion, and a distal tip. A plurality of electroanatomical mapping electrodes are located on the pre-formed distal portion. The plurality of electroanatomical mapping electrodes are configured to electrically couple with an electroanatomical mapping system. The pre-formed distal portion has a substantially linear configuration when confined in the sheath, and an arcuate configuration when unconfined.

Example 32 is the system of Example 31 wherein the pre-formed distal portion includes a first curve, a second curve, and a medial segment between the first curve and the second curve, and wherein an axis of the tapered distal tip is offset from an axis of the medial segment when the pre-formed distal portion is in the arcuate configuration.

Example 33 is the system of Example 31 wherein the pre-formed distal portion and the distal tip are located in the same plane when the pre-formed distal portion is in the arcuate configuration.

Example 34 is the system of Example 31 wherein the pre-formed distal portion and the distal tip are located in different planes when the pre-formed distal portion is in the arcuate configuration.

Example 35 is a medical method. The method includes inserting a sheath and dilator into the right atrium. The dilator is advanced from a distal tip of the sheath into a mapping configuration. The method includes moving the dilator around the right atrium to generate an electroanatomical map of the right atrium. The dilator is retracted into the sheath to adopt a transseptal crossing configuration. The fossa ovalis is punctured via a piercing device that is inserted through a hollow lumen of the dilator. The method includes advancing the sheath and dilator into the left atrium of the heart.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 illustrates a sheath and shaped dilator with EAM electrodes in accordance with an embodiment of the present disclosure.

FIGS. 3A and 3B illustrate various configurations for shaped dilator in accordance with embodiments of the present disclosure.

FIG. 4 illustrates the sheath and shaped dilator of FIG. 2 configured in a mapping configuration in a left atrium in accordance with an embodiment of the present disclosure.

FIG. 5 illustrates the sheath and shaped dilator of FIG. 2 configured in a transseptal crossing configuration placed against an atrial septum in accordance with an embodiment of the present disclosure.

FIG. 6 illustrates the sheath and shaped dilator of FIG. 2 configured in a mapping configuration in a right atrium in accordance with an embodiment of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

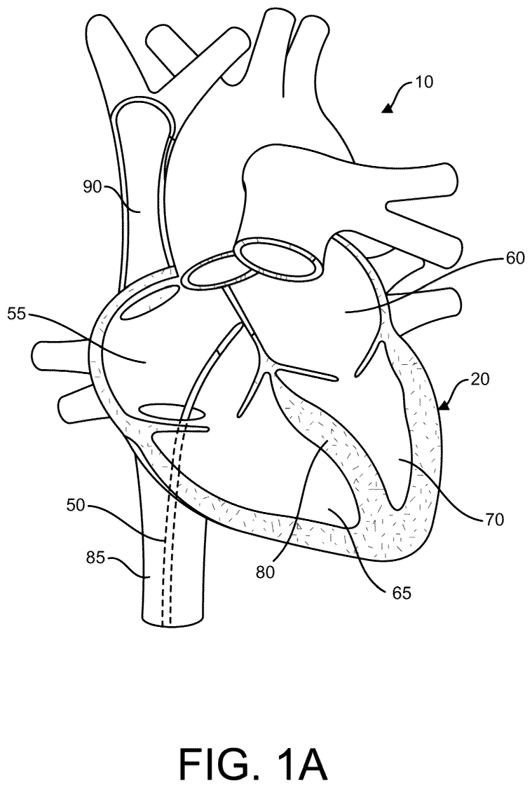

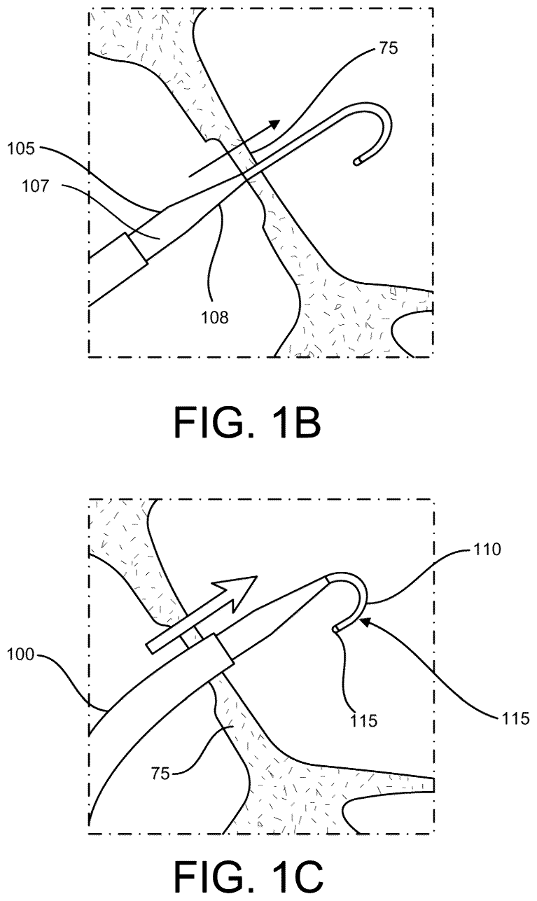

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In some aspects, the guidewire may itself include an RF electrode so as to function as an RF perforation device.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 200 V RMS (565 V peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patents application Ser. No. 16/445,790 and Ser. No. 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In some aspects, the RF perforation device 110 may be replaced with a mechanical piercing device such as a needle having a sharp distal tip. The needle can be configured to pierce the atrial septum 75 when the sharp distal tip is positioned on the atrial septum 75 and pressure is applied to the proximal end.

In some aspects, it may be desirable for the dilator 105, sheath 100, or RF perforation device 110 to include one or more surface electrode. The one or more surface electrode may be located on a distal portion of the dilator 105, sheath 100, or RF perforation device 110 for use in ablation, mapping, pacing, or sensing a parameter within a portion of the heart 20. The one or more surface electrode may be connected to an electroanatomical mapping (EAM) system, generator, or other diagnostic system.

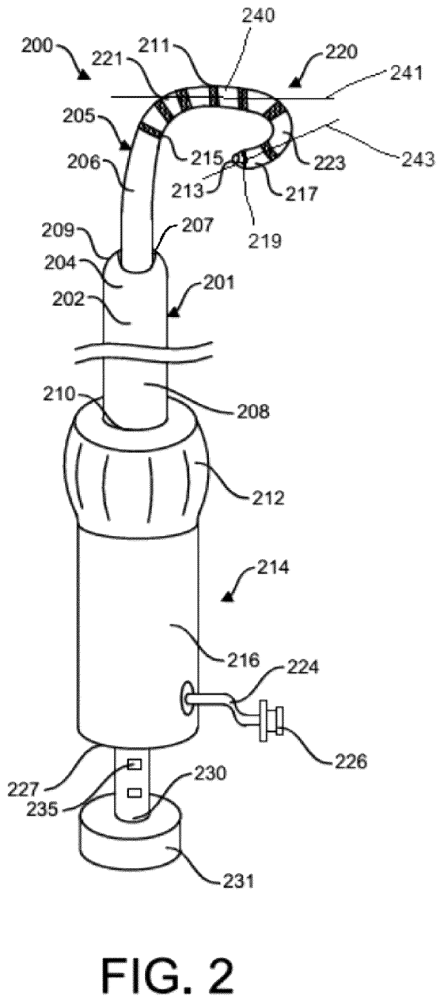

FIG. 2 illustrates a system 200 including a sheath 201 and shaped dilator 205 in accordance with an embodiment of the present disclosure. The sheath 201 includes an elongate hollow body 202 having a proximal portion 208 and a distal portion 204. The distal portion 204 ends in a distal tip 209.

The proximal portion 208 includes a proximal end 210 that removably couples to a handle 214. The handle 214 includes a stationary portion 216 configured to be held by a hand of a user and a rotatable knob 212 that is configured to adjust the shape of the distal portion 204. Rotatable knob 212 is connected to at least one control wire configured to deflect the distal portion 204 in a first direction when the knob 212 is rotated clockwise and to deflect the distal portion 204 in a second direction when the knob 212 is rotated counterclockwise. In some aspects, a dial, plunger, or sliding mechanism may be used instead of the rotatable knob 212 to control one or more pull wire or push rod to deflect a portion of the sheath 201.

A lumen 207 extends from a handle proximal end 227 though the knob 212 and through the hollow body 202 to the distal tip 209. The lumen 207 is configured to receive a dilator 205, other elongate medical device, or fluids.

The stationary portion 216 includes a conduit 224 which includes a fitting 226, such as a Luer connector. The conduit 224 is configured to allow for introduction of fluids into the lumen 207 or an additional lumen within the sheath 201. The fitting 226 is configured to removably connect to a syringe or other container for delivering a fluid through the conduit 224. In some aspects, the sheath 201 may include one or more electrode. In this situation, the stationary portion 216 could include a cable configured to couple with a system, for example an EAM system or RF energy generator. The cable would include a connector that releasably engages with a connector for the system.

The shaped dilator 205 is configured to be movable within the lumen 207. The shaped dilator 205 includes an elongate hollow body 206 having a proximal portion and a distal portion. The proximal portion includes a proximal end 230 removably coupled to a hub or handle 231. In some aspects, the hub or handle 231 may include a steering mechanism to allow for changing the shape of the distal portion. The hub or handle 231 allows a user to extend and withdrawal the shaped dilator 205 from and into the sheath 201.

The distal portion includes a tapered section 217, a distal end 213, and an arcuate portion 220. The tapered section 217 tapers from an outer diameter of approximately 0.111″ to approximately 0.060″ over a length of approximately 10 mm. The arcuate portion 220 is pre-formed such that upon exiting from the sheath 201 in a mapping configuration, the arcuate portion 220 assumes a shape such that the tapered section 217 and the distal end 213 generally point at the elongate hollow body 206. The arcuate portion 220 is more flexible than the elongate hollow body 202 of the sheath such that when constrained within the lumen 207 of the sheath 201, the arcuate portion 220 remains substantially linear and takes the shape of the elongate hollow body 202.

In various embodiments, the arcuate portion 220 has a circular shape, such that the distal end 213 is positioned substantially adjacent the hollow body 206. The arcuate portion 220 includes medial segment 240 located between a first curve 221 and a second curve 223. In some aspects, the arc length and the radius of the first curve 221 and the second curve 223 are such that an axis 243 of the tapered section 217 is offset from an axis 241 of the medial segment 240 when in a mapping configuration. In some aspects, the arc length and the radius of the first curve 221 and the second curve 223 are such that the tapered section 217 and the distal end 213 generally point at the elongate hollow body 206 when in a mapping configuration. It is understood that the arc length and the radius of each of the first curve 221 and the second curve 223 can be changed to alter the configuration of the arcuate portion 220 when in a mapping configuration. For example, the arcuate portion 220 may have a consistent curvature in one aspect, leading to a generally circular shape. In another aspect, the arcuate portion 220 may include various curvature, leading to a generally oval shape or a generally spiral shape.

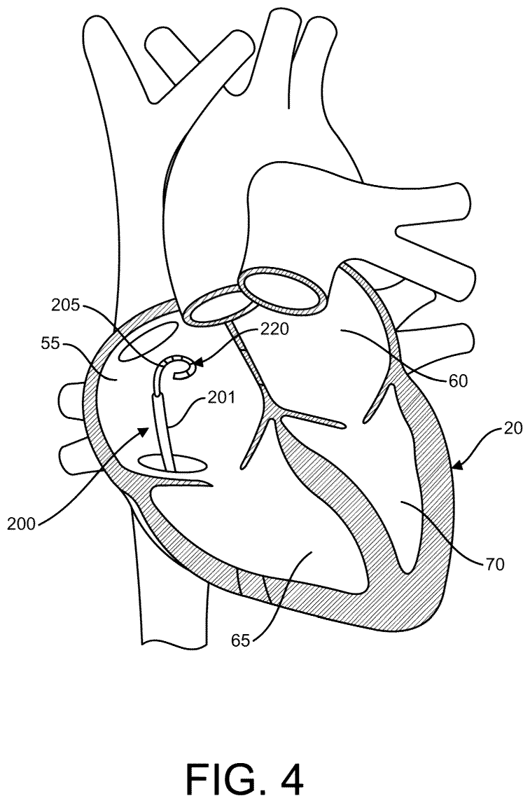

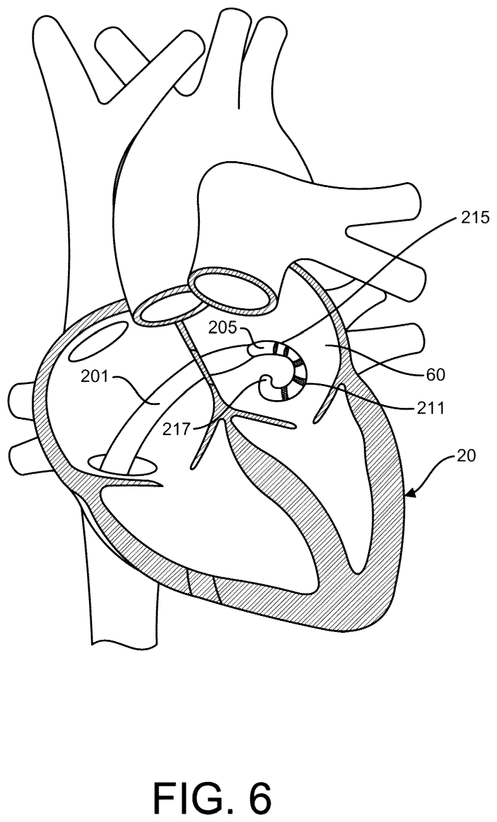

FIG. 4 illustrates the sheath 201 and shaped dilator 205 in a mapping configuration in a right atrium of a patent. FIG. 6 illustrates the sheath 201 and shaped dilator 205 in a mapping configuration in a left atrium of a patient.

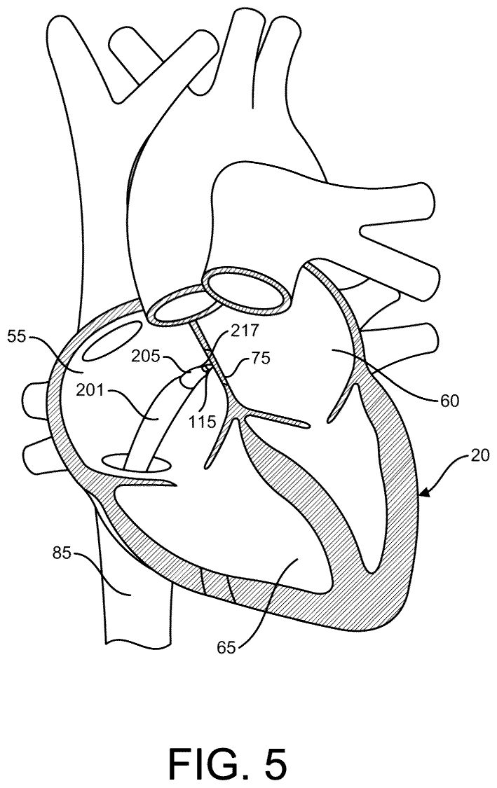

A distal portion of the arcuate portion 220, situated just proximal of the tapered section 217, is substantially straight to ensure appropriate configuration when the dilator is in a transseptal crossing configuration. FIG. 5 illustrates the sheath 201 and shaped dilator 205 in a transseptal crossing configuration placed against an atrial septum 75 in accordance with an embodiment of the present disclosure. In the transseptal crossing configuration, the arcuate portion 220 of the shaped dilator 205 is withdrawn into the sheath 201 such that only the substantially straight portion and the tapered section 217 are exposed.

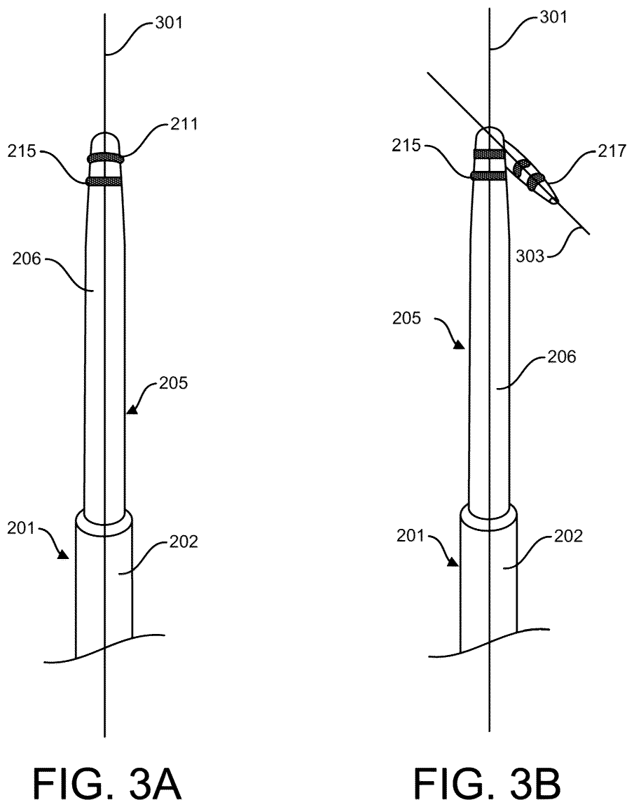

In one aspect, illustrated in FIG. 3A, the distal portion of the shaped dilator 205 remains in a single plane illustrated at 301 when extended out of the sheath 201. In this configuration, the arcuate portion 220 and the tapered section 217 share a common plane. In another aspect, illustrated in FIG. 3B, the distal portion of the shaped dilator 205 extends in more than one plane. For example, the arcuate portion 220 may remain in a first plane illustrated at 301 while the tapered section 217 extends in a second plane illustrated at 303. While two planes are shown, it is understood that the distal portion of the shaped dilator 205 may extend in three or more planes when unconfined by the sheath 201. In some aspects, one or more control wire or push rod may be used to change the shape of the arcuate portion 220. In other aspects, the arcuate portion 220 may have a pre-formed curve that causes the distal portion to extend in three or more planes. In some aspects, the arcuate portion 220 may include an embedded nitinol shaping wire or may include a laser-cut hypotube that allows the arcuate portion 220 to obtain the pre-formed curve when unconfined by the sheath 201.

The distal portion includes one or more electrodes 211. The one or more electrodes 211 can be evenly spaced or positioned at uneven intervals. The one or more electrodes 211 can be configured as surface electrodes capable of contacting tissue or fluid within a patient. The one or more electrodes 211 may be configured as ring electrodes formed of a single conductive material, or from a plurality of materials. The one or more electrodes 211 may be solid, formed by doping, electroplating, or other method for creating an electrically conductive electrode. The one or more electrodes 211 may be configured to detect a parameter when in contact with the tissue or fluid or to deliver energy to the tissue, such as RF energy in order to ablate the tissue. The one or more electrodes 211 can be electrically coupled to a control system, such as an electroanatomical mapping system or an energy generator.

In some aspects, the one or more electrodes 211 may be no smaller than 1 mm in width and no larger than 5 mm in width. While a plurality of electrodes are illustrated, in some applications only a single electrode may be required for creating rudimentary map of a patient's heart. The one or more electrodes 211 are connected to wires or conductors traversing the length of the shaped dilator 205 to interface with a control system, such as an electroanatomical mapping system. The shaped dilator 205 may include one or more lumens to accommodate the wires or conductors for each of the one or more electrodes 211.

The one or more electrodes 211 includes a proximal electrode 215 and a tip electrode 219. The proximal electrode 215 is located proximal of the first curve 221. This allows a mapping system to identify the beginning of the arcuate portion 220 and display the configuration of the shaped dilator 205 on a display. Visualization of the proximal electrode 215 ensures that the arcuate portion 220 is completely extending from the sheath 201.The tip electrode 219 is located at the distal end 213 of the shaped dilator 205.

The shaped dilator includes a lumen that extends from the hub or handle 231 through the elongate hollow body 206 to the distal end 213. The lumen is configured to receive an elongate medical device, such as a guidewire or piercing or perforation device. Fluids may also be inserted into the lumen from the hub or handle 231.

The shaped dilator 205 can have a lumen diameter in the range of approximately 032″-0.060″ and may be formed of a variety of materials, including low density polyethylene (LDPE) and high density polyethylene (HDPE).

The proximal portion of the shaped dilator 205 may include one or more indicators 235. The one or more indicator 235 gives an indication for when the dilator 205 is in mapping and transseptal crossing configurations. The one or more indicators 235 can be a visual (e.g. visual marker) and/or tactile (e.g. bumps/grooves) means configured to indicate to user when the shaped dilator 205 is in a mapping configuration or a transseptal crossing configuration. The one or more indicators 235 may also indicate when the shaped dilator 205 is completely withdrawn into the sheath 201.

FIG. 4 illustrates the sheath 201 and shaped dilator 205 of FIG. 2 configured in a mapping configuration in a right atrium 55 of a patient's heart 20 in accordance with an embodiment of the present disclosure. In FIG. 4, the sheath 201 and shaped dilator 205 have been placed within a right atrium 55 using a guidewire or other guiding member. The shaped dilator 205 is illustrated as being advanced out of the distal end of the sheath 20 into a mapping configuration. In the mapping configuration, the arcuate section 220 of the shaped dilator 205 adopts a pre-formed arcuate configuration. The one or more electrodes 211 are connected to an electroanatomical mapping system and are used to create an electroanatomical map of the right atrium 55 as the system 200 is moved throughout the right atrium 55. The sheath 201 and/or dilator 205 may be equipped with a steering system that enhances movement of the dilator 205 in order to create the electroanatomical map.

Following creation of an electroanatomical map of the right atrium 55, the shaped dilator 205 can be withdrawn into the sheath 201 until the pre-formed curve of the arcuate section 220 that was present in the mapping configuration conforms to the sheath 201. This configuration is useful for performing a transseptal puncture. FIG. 5 illustrates the sheath 201 and shaped dilator 205 of FIG. 2 configured in a transseptal crossing configuration with the distal end of the dilator 205 placed against an atrial septum 75 in accordance with an embodiment of the present disclosure. Once positioned, a puncturing device may be advanced though the lumen of the dilator 205 to perform the transseptal puncture immediately after creating a map of the right atrium 55. FIG. 5 illustrates the tip electrode 115 of a puncturing device prior to piercing the atrial septum 75.

FIG. 6 illustrates the sheath 201 and shaped dilator 205 of FIG. 2 configured in a mapping configuration in a left atrium 60 in accordance with an embodiment of the present disclosure. Following the transseptal puncture, the sheath 201 and shaped dilator 205 are advanced into the left atrium 60. The shaped dilator 205 is re-advanced from the distal end of the sheath 201 into a mapping configuration. In this position, an electroanatomical map of the left atrium 60 may be created using the one or more electrodes 211 of the dilator 205 and moving the dilator 205 around the left atrium.

In some aspects, a control system, for example an EAM system or energy generator, may include a display for displaying one or more anatomical images, parameters, positioning information, 3-D anatomical rendering of the relevant cardiac chambers, and medical device information.

In some aspects, the sheath 201 and shaped dilator 205 can be packaged as a kit and be ready for use right out of the package. The kit may also include a RF perforation or puncturing device 110. Alternatively, the kit may include a plurality of sheaths or dilators each having different pre-shaped portions for use in various procedures.

In some aspects, the sheath 201 or shaped dilator 205 may include one or more markers along a distal portion thereof for identifying a position or location during use using an imaging modality.

In some aspects, the sheath 201 or dilator 205 may include a plurality of cuts machined into a wall thereof, for example by laser cutting. The shape and positioning of the cuts can allow for a transition in flexibility from a proximal portion to distal portion. The cuts may include a broken spiral configuration or may be positioned substantially orthogonal to a longitudinal axis of the sheath 201 or dilator 205. In some aspects, there may be a single cut that winds around an axis with a wider spacing between loops at the proximal portion and a larger spacing at the distal portion. The spacing and size of the cuts can be varied to achieve different flexibilities along the length of the sheath 201 or dilator 205.

In some aspects, the sheath 201 or dilator 205 may be formed of a shape memory material, such as a shape memory polymer or a shape memory metal. This would allow the sheath 201 or dilator 205 to have a first shape at a first temperature, and a second shape at a second temperature. Shape transition may be initiated by inserting a heated solution into the sheath 201 or dilator 205 or using electricity to heat a portion of the sheath 201 or dilator 205.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A medical system, comprising:
a sheath including an elongate body having a proximal portion and a distal portion, and a lumen extending from the proximal portion to the distal portion; and
a dilator movable within the lumen, the dilator including an elongate body having a proximal portion, a pre-formed distal portion, a tapered distal tip, and a lumen extending from the proximal portion to the tapered distal tip;
one or more electroanatomical mapping electrodes located on the pre-formed distal portion, the one or more electroanatomical mapping electrodes being configured to electrically couple with an electroanatomical mapping system;
wherein the pre-formed distal portion has a substantially linear configuration when confined in the sheath, and an arcuate configuration when unconfined.

2. The system of claim 1, wherein the sheath or the dilator includes a steering mechanism.

3. The system of claim 1, wherein the pre-formed distal portion includes a first curve, a second curve, and a medial segment between the first curve and the second curve, and wherein an axis of the tapered distal tip is offset from an axis of the medial segment when the pre-formed distal portion is in the arcuate configuration.

4. The system of claim 1, wherein the pre-formed distal portion and the tapered distal tip are located in the same plane when the pre-formed distal portion is in the arcuate configuration.

5. The system of claim 1, wherein the pre-formed distal portion and the tapered distal tip are located in different planes when the pre-formed distal portion is in the arcuate configuration.

6. The system of claim 1, wherein the sheath has a rigidity greater than a rigidity of the pre-formed distal portion.

7. The system of claim 1, further comprising a guiding member configured to be inserted into the lumen of the dilator.

8. The system of claim 1, further comprising a piercing member configured to be inserted into the lumen of the dilator.

9. The system of claim 8, wherein the piercing member is RF perforation device or a needle.

10. The system of claim 1, wherein one or more conductor extends from the one or more electroanatomical mapping electrodes to the proximal portion of the dilator elongate body.

11. The system of claim 1, wherein the tapered distal tip tapers from an outer diameter of approximately 0.111″ to approximately 0.060″ over a length of approximately 10 mm.

12. The system of claim 1, wherein the dilator is formed of one or more low density polyethylene, high density polyethylene, shape memory polymer, or shape memory metal.

13. The system of claim 1, further comprising a proximal electrode located proximal of the pre-formed distal portion that is configured to identify a stem of the dilator on the electroanatomical mapping system.

14. The system of claim 1, wherein the electroanatomical mapping system comprises a display for displaying one or more anatomical images, parameters, and positioning information.

15. The system of claim 1, wherein the one or more electroanatomical mapping electrodes includes three electrodes.

16. A medical system, comprising:
a sheath including an elongate body having a proximal portion and a distal portion, and a lumen extending from the proximal portion to the distal portion; and
a dilator movable within the lumen, the dilator including an elongate body having a proximal portion, a pre-formed distal portion, and a distal tip;
a plurality of electroanatomical mapping electrodes located on the pre-formed distal portion, the plurality of electroanatomical mapping electrodes being configured to electrically couple with an electroanatomical mapping system;
wherein the pre-formed distal portion has a substantially linear configuration when confined in the sheath, and an arcuate configuration when unconfined.

17. The system of claim 16, wherein the pre-formed distal portion includes a first curve, a second curve, and a medial segment between the first curve and the second curve, and wherein an axis of the tapered distal tip is offset from an axis of the medial segment when the pre-formed distal portion is in the arcuate configuration.

18. The system of claim 16, wherein the pre-formed distal portion and the distal tip are located in the same plane when the pre-formed distal portion is in the arcuate configuration.

19. The system of claim 16, wherein the pre-formed distal portion and the distal tip are located in different planes when the pre-formed distal portion is in the arcuate configuration.

20. A medical method, comprising:
inserting a sheath and dilator into the right atrium;
advancing the dilator from a distal tip of the sheath into a mapping configuration;
moving the dilator around the right atrium to generate an electroanatomical map of the right atrium;
retracting the dilator into the sheath to adopt a transseptal crossing configuration;
puncturing the fossa ovalis via a piercing device that is inserted through a hollow lumen of the dilator; and
advancing the sheath and dilator into the left atrium of the heart.
